(19) United States
(12) Patent Application Publication
(10) Pub. No.: US 2024/0350702 A1
(43) Pub. Date: Oct. 24, 2024

(54) ANTIBACTERIAL DRESSING FOR PROMOTING SCARLESS HEALING OF WOUND SURFACE AND METHOD FOR PREPARING THE SAME

(71) Applicants: ZHEJIANG UNIVERSITY, Hangzhou (CN); SHAOXING RESEARCH INSTITUTE OF ZHEJIANG UNIVERSITY, Shaoxing (CN)

(72) Inventors: Changyou GAO, Hangzhou (CN); Jie WANG, Hangzhou (CN); Xiaofei DONG, Hangzhou (CN)

(21) Appl. No.: 18/757,611

(22) Filed: Jun. 28, 2024

Related U.S. Application Data
(63) Continuation of application No. PCT/CN2022/097163, filed on Jun. 6, 2022.

(30) Foreign Application Priority Data
May 30, 2022 (CN) 202210600635.X

(51) Int. Cl. A61L 26/00

(52) U.S. Cl. CPC A61L 26/0014; A61L 26/0023; A61L 26/008; A61L 2300/404

(57) ABSTRACT

An antibacterial dressing for promoting scarless wound healing and a method for preparing the same provided. The dressing consists of 0.05-0.1 part by weight of hyperbranched polylysine anti-scar compound, 0.1-2.0 parts by weight of carbomer, 1-10 parts by weight of a thickener, 2-12 parts by weight of a humectant, 60-90 parts by weight of water and 1-15 parts by weight of a plant extract. The hyperbranched polylysine and the anti-scar drug are grafted and synthesized into a new compound, so that the antibacterial activity of the hyperbranched polylysine and the ability of the anti-scar drug to inhibit the scar formation of the wound are retained; meanwhile, a film with good air permeability and moisture retention can be formed on the skin surface through the film forming agent, so that the wound surface is kept in a moist environment, which is beneficial to wound healing and does not generate scars.

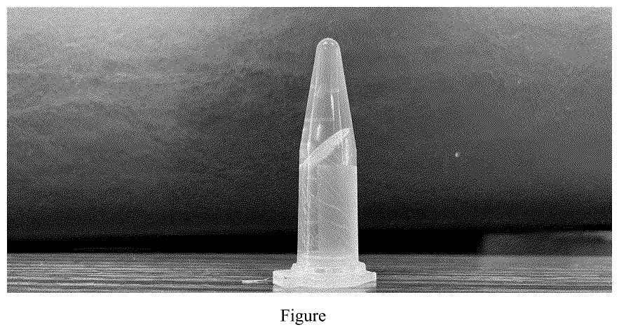

### TECHNICAL FIELD

The present application relates to an antibacterial dressing for promoting scarless wound healing and a method for preparing the same, belonging to the technical field of biomedicine.

### BACKGROUND

Skin is the largest organ of human body, and it is a necessary interface to connect the internal and external environment. Therefore, it continuously protects the body from harmful attacks from the outside world, so it is also an easily injured organ of the human body. The injured skin will inevitably contact with various pathogenic microorganisms in the environment. If infection occurs before the wound is closed, it will prolong the healing time and lead to the death of the patient. Therefore, antibacterial property is one of the most important properties of dressings.

Scar is the result of the interaction of inflammation, proliferation and tissue remodeling in the healing stage of skin wound. Although the mechanism of scar formation is still unclear, it is generally believed that excessive inflammation during wound healing will lead to scar. Pathological scar is a pathological result caused by various internal and external factors in bed, including hypertrophic scar and keloid, which is characterized by excessive deposition of a large number of extracellular matrix such as collagen and excessive proliferation of fibroblasts. When the scar grows beyond a certain limit, it will produce various complications, such as the destruction of appearance and dysfunction, which will bring great physical pain and mental pain to patients, especially the scars left after burns, scalds and serious injuries, which are difficult to eliminate if measures are not taken. A few years of scar hyperplasia made the patients miserable, and then the atrophic period made the patients unrecognizable and dysfunctional, causing great physical and mental harm to the patients. Therefore, effectively inhibiting scar formation is also one of the most important properties of dressing.

At present, the main methods to treat scars are applying scar cream, wearing elastic clothes, superficial radiotherapy, injection therapy, laser therapy and surgical treatment. These methods have their own advantages and disadvantages, but none of them can achieve perfect results. At present, most dressings on the market are silicone hydrogels made of single polysiloxane in different proportions or by reducing the level of inflammation to prevent and treat scars, so that water evaporation is reduced, skin moisture is only left in the stratum corneum, interstitial water-soluble proteins and low-molecular-weight water-soluble substances are diffused to the surface, interstitial water-soluble substances are reduced, and scar tissue is softened. However, the function of silicone hydrogel is single, and it can only be used to close wounds, so the treatment effect is not good. The effect of preventing scars by reducing the level of inflammation alone is also limited. CN 112704691A discloses a Chinese medicinal ointment for promoting wound healing and reducing scar formation, which can reduce the level of inflammation during wound healing and has certain antibacterial effect. However, it cannot effectively provide a moist environment for the wound. According to the “moist healing theory”, creating a moist healing environment close to the physiological state during wound care is conducive to the growth of granulation and the division of skin cells, thus promoting the complete healing of the wound.

### SUMMARY

One of the objects of the present application is to provide a hydrogel dressing that can be used to promote wound closure, which can provide a good wet environment for wound healing; the second object of the present application is to provide an antibacterial hydrogel dressing used to promote wound closure, which can provide a moist environment for wound healing, at the same time, it can also have a broad-spectrum antibacterial effect, and has an excellent killing effect on common pathogenic bacteria, avoiding wound infection and promoting wound healing; the third object of the present application is to provide an antibacterial hydrogel dressing which can be used to promote wound closure and scarless healing. In addition, active components in the dressing have the effects of relieving inflammation and targeting the downstream proteins and other cell pathways related to scar formation, which can effectively promote scarless healing of wounds.

One of the objects of the present application is achieved through the following technical solution.

An antibacterial dressing for promoting scarless wound healing, includes the following raw materials in parts by mass:
0.05-0.1 part of a hyperbranched polylysine anti-scar compound, 0.1-2.0 parts of carbomer, 1-10 parts of a thickener, 2-12 parts of a humectant, 60-90 parts of water and 1-10 parts of a plant extract, and the pH is regulated to 6.8-7.2 with alkali.

Preferably, the plant extract is at least one of aloe extract and cactus extract.

Preferably, the humectant is glycerol.

Preferably, the thickener is one or more of polyvinyl alcohol, hydroxypropyl methylcellulose, hydroxyethyl cellulose and polyvinylpyrrolidone.

Preferably, the carbomer is a series of carbomer products, including at least one of carbomer 910, carbomer 934, carbomer 934P, carbomer 940 and carbomer 941.

Preferably, the water is deionized water.

A method for preparing an antibacterial dressing for promoting scarless wound healing includes the following steps:
(1) adding 1-10 parts of the thickener and 2-12 parts of the humectant into 60-90 parts of deionized water, magnetically stirring and dissolving uniformly, then adding 1-10 parts of the plant extract, and fully stirring and dissolving in vacuum for later use;
(2) adding 0.1-2.0 parts of carbomer into 90-100 parts of water for vacuum magnetic stirring and dissolution;
(3) uniformly mixing the solutions prepared in (1) and (2) above, and adding an alkali solution to adjust pH to 6.8-7.2, so as to obtain a hydrogel dressing which can be used on an unclosed wound, hereinafter referred to as a blank hydrogel dressing.

The second object of the present application is realized by the following technical solution:
adding hyperbranched polylysine to the blank hydrogel dressing, and magnetically stirring and mixing uniformly in vacuum to obtain an antibacterial hydrogel dressing for promoting wound healing.

The third object of the present application is realized by the following technical solution:
(1) taking a certain amount of hyperbranched polylysine to prepare an aqueous solution of 10-30 mg/mL; mixing a fresh stock solution of 10-40 mg/ml of 2-iminothiolane hydrochloride (2-IT) with hyperbranched polylysine solution in a volume ratio of 3:1, and reacting at room temperature for two hours; performing rotary evaporation on the reaction solution, repeating washing with isopropanol and rotary evaporation, and drying in a vacuum oven until the mass is constant (25° C.); then dissolving the obtained thiol-modified hyperbranched polylysine and the anti-scar drug (asiaticoside or salvianolic acid B or papain) in water in a mass ratio of 1:1 to prepare a solution of 10-20 mg/mL, reacting at 50° C. for 5 hours, performing rotary evaporation on the reaction solution, repeating washing with isopropanol and rotary evaporation, and then drying in a vacuum oven until the mass is constant (25° C.), to finally obtain the hyperbranched polylysine anti-scar compound;
(2) weighing by mass 1-30 parts of hyperbranched polylysine and 1-30 parts of the anti-scar drug, adding into 100 parts of deionized water, and stirring until completely dissolved to obtain a mixed solution; weighing 0.6 part of 1-ethyl-(3-dimethylaminopropyl) carbodiimide (EDC) and 0.3 part of N-hydroxysuccinimide (NHS), adding into the above mixed solution, then adjusting the pH to 5.5 with 2-(N-morpholine) ethylenesulfonic acid (NHS), and magnetically stirring at room temperature for reaction for 5-10 hours; then, subjecting the reaction solution to rotary evaporation, and repeating washed with isopropanol and rotary evaporation, and finally drying the obtained product in a vacuum oven until the mass is constant (25° C.), to finally obtain the hyperbranched polylysine anti-scar compound.

Then, preparing the prepared hyperbranched polylysine anti-scar compound into an aqueous solution, and adding into the blank hydrogel dressing, and magnetically stirring and mixing uniformly in vacuum, to obtain an antibacterial hydrogel dressing for promoting scarless wound healing.

The present application has the beneficial effects that:

According to the present application, a brand-new compound is synthesized by grafting hyperbranched polylysine and an anti-scar drug by a chemical method, and a film with good air permeability and moisture retention can be formed on the skin surface through the film forming agent, so that the wound surface is kept in a moist environment, which is beneficial to wound healing and does not generate scars.

### BRIEF DESCRIPTION OF DRAWINGS

FIGURE shows the hydrogel dressing prepared by the present application.

### DESCRIPTION OF EMBODIMENTS

In the following, the present application will be further described in combination with specific embodiments. It should be noted that, under the premise of no conflict, the following examples or technical features can be arbitrarily combined to form a new example.

### Example 1

An aqueous solution of 30 mg/mL was prepared from a certain amount of hyperbranched polylysine, a fresh stock solution of 30 mg/mL 2-iminothiolane hydrochloride (2-IT) was mixed with the hyperbranched polylysine solution according to a volume ratio of 3:1, and the reaction solution was subjected to mild reaction for two hours at room temperature; the reaction solution was subjected to rotary evaporation and repeated washing with isopropanol, and then dried in a vacuum oven until the mass was constant (25° C.); then the obtained thiol-modified hyperbranched polylysine and the anti-scar drug, asiaticoside, were dissolved in water according to a mass ratio of 1:1 to prepare a solution of 15 mg/mL, which was reacted at 50° C. for 5 hours; the reaction solution was subjected to rotary evaporation and repeated washing with isopropanol, and then dried in a vacuum oven until the mass was constant (25° C.), and finally a hyperbranched polylysine anti-scar compound was obtained.

3 parts by weight of an aloe extract, 7 parts by weight of a cactus extract, 15 parts by weight of a thickener and 0.1 part by weight of the prepared hyperbranched poly-lysine anti-scar compound were weighed respectively, and then 10 parts by weight of glycerol and 80 parts by weight of deionized water were weighed, and were fully stirring and dissolving uniformly for later use; 2.0 parts by weight of carbomer 940 were added into 80 parts by weight of water, and heated to 60° C. to fully dissolve carbomer 940 in water, and then cooled to room temperature, poured into the prepared thickener solution, and the mixture was fully stirred and dissolved; finally a proper amount of a pH regulator was added to adjust the pH to 6.8-7.2, so as to obtain an antibacterial hydrogel dressing for promoting scarless wound healing; its appearance is shown in the FIGURE, and the hydrogel dressing prepared by the present application is a non-flowable hydrogel.

### Example 2

An aqueous solution of 30 mg/mL was prepared from a certain amount of hyperbranched polylysine, a fresh stock solution of 30 mg/mL 2-iminothiolane hydrochloride (2-IT) was mixed with the hyperbranched polylysine solution according to a volume ratio of 3:1, and the reaction solution was subjected to mild reaction for two hours at room temperature; the reaction solution was subjected to rotary evaporation and repeated washing with isopropanol, and then dried in a vacuum oven until the mass was constant (25° C.); then the obtained thiol-modified hyperbranched polylysine and the anti-scar drug, salvianolic acid B, were dissolved in water according to a mass ratio of 1:1 to prepare a solution of 15 mg/mL, which was reacted at 50° C. for 5 hours; the reaction solution was subjected to rotary evaporation and repeated washing with isopropanol, and then dried in a vacuum oven until the mass was constant (25° C.), and finally a hyperbranched polylysine anti-scar compound was obtained.

3 parts by weight of an aloe extract, 7 parts by weight of a cactus extract, 15 parts by weight of a thickener and 0.1 part by weight of the prepared hyperbranched poly-lysine anti-scar compound were weighed respectively, and then 10 parts by weight of glycerol and 80 parts by weight of deionized water were weighed, and were fully stirring and dissolving uniformly for later use; 2.0 parts by weight of carbomer 940 were added into 80 parts by weight of water, and heated to 60° C. to fully dissolve carbomer 940 in water, and then cooled to room temperature, poured into the prepared thickener solution, and the mixture was fully stirred and dissolved; finally a proper amount of a pH regulator was added to adjust the pH to 6.8-7.2, so as to obtain an antibacterial hydrogel dressing for promoting scarless wound healing.

### Example 3

An aqueous solution of 30 mg/mL was prepared from a certain amount of hyperbranched polylysine, a fresh stock solution of 30 mg/mL 2-iminothiolane hydrochloride (2-IT) was mixed with the hyperbranched polylysine solution according to a volume ratio of 3:1, and the reaction solution was subjected to mild reaction for two hours at room temperature; the reaction solution was subjected to rotary evaporation and repeated washing with isopropanol, and then dried in a vacuum oven until the mass was constant (25° C.); then the obtained thiol-modified hyperbranched polylysine and the anti-scar drug, papain, were dissolved in water according to a mass ratio of 1:1 to prepare a solution of 15 mg/mL, which was reacted at 50° C. for 5 hours; the reaction solution was subjected to rotary evaporation and repeated washing with isopropanol, and then dried in a vacuum oven until the mass was constant (25° C.), and finally a hyperbranched polylysine anti-scar compound was obtained.

3 parts by weight of an aloe extract, 7 parts by weight of a cactus extract, 15 parts by weight of a thickener and 0.1 part by weight of the prepared hyperbranched poly-lysine anti-scar compound were weighed respectively, and then 10 parts by weight of glycerol and 80 parts by weight of deionized water were weighed, and were fully stirring and dissolving uniformly for later use; 2.0 parts by weight of carbomer 940 were added into 80 parts by weight of water, and heated to 60° C. to fully dissolve carbomer 940 in water, and then cooled to room temperature, poured into the prepared thickener solution, and the mixture was fully stirred and dissolved; finally a proper amount of a pH regulator was added to adjust the pH to 6.8-7.2, so as to obtain an antibacterial hydrogel dressing for promoting scarless wound healing.

### Example 4

30 parts of hyperbranched polylysine and 30 parts of asiaticoside were weighed to be added into 100 parts of deionized water, and stirred until they are completely dissolved to obtain a mixed solution; 0.6 part of 1-ethyl-(3-dimethylaminopropyl) carbodiimide (EDC) and 0.3 part of N-hydroxysuccinimide (NHS) were weighed to be added into the above mixed solution, then the pH was adjusted to 5.5 with 2-(N-morpholine) ethylenesulfonic acid (NHS), and magnetic stirring was carried out for reaction at room temperature for 5-10 hours; then, the reaction solution was subjected to rotary evaporation and repeatedly washing with isopropanol, and a final product was dried in a vacuum oven until the mass was constant (25° C.), and finally the hyperbranched polylysine anti-scar compound was obtained.

3 parts of the aloe extract, 7 parts of the cactus extract, 15 parts of the thickener, 0.1 part of the hyperbranched polylysine anti-scar compound prepared above, 10 parts of glycerol and 80 parts of deionized water were weighed respectively and were fully stirred and dissolved uniformly for later use; 2.0 parts of carbomer 940 were added into 80 parts of water, the mixture was heated to 60° C. to fully dissolve carbomer 940 in water, and then cooled to room temperature, poured into the prepared thickener solution, and the mixture was fully stirred and dissolved; finally a proper amount of a pH regulator was added to adjust the pH to 6.8-7.2, so as to obtain an antibacterial hydrogel dressing for promoting scarless wound healing.

### Example 5

30 parts of hyperbranched polylysine and 30 parts of salvianolic acid B were weighed to be added into 100 parts of deionized water, and stirred until they are completely dissolved to obtain a mixed solution; 0.6 part of 1-ethyl-(3-dimethylaminopropyl) carbodiimide (EDC) and 0.3 part of N-hydroxysuccinimide (NHS) were weighed to be added into the above mixed solution, then the pH was adjusted to 5.5 with 2-(N-morpholine) ethylenesulfonic acid (NHS), and magnetic stirring was carried out for reaction at room temperature for 5-10 hours; then, the reaction solution was subjected to rotary evaporation and repeatedly washing with isopropanol, and a final product was dried in a vacuum oven until the mass was constant (25° C.), and finally the hyperbranched polylysine anti-scar compound was obtained.

3 parts of the aloe extract, 7 parts of the cactus extract, 15 parts of the thickener, 0.1 part of the hyperbranched polylysine anti-scar compound prepared above, 10 parts of glycerol and 80 parts of deionized water were weighed respectively and were fully stirred and dissolved uniformly for later use; 2.0 parts of carbomer 940 were added into 80 parts of water, the mixture was heated to 60° C. to fully dissolve carbomer 940 in water, and then cooled to room temperature, poured into the prepared thickener solution, and the mixture was fully stirred and dissolved; finally a proper amount of a pH regulator was added to adjust the pH to 6.8-7.2, so as to obtain an antibacterial hydrogel dressing for promoting scarless wound healing.

### Example 6

30 parts of hyperbranched polylysine and 30 parts of papain were weighed to be added into 100 parts of deionized water, and stirred until they are completely dissolved to obtain a mixed solution; 0.6 part of 1-ethyl-(3-dimethylaminopropyl) carbodiimide (EDC) and 0.3 part of N-hydroxysuccinimide (NHS) were weighed to be added into the above mixed solution, then the pH was adjusted to 5.5 with 2-(N-morpholine) ethylenesulfonic acid (NHS), and magnetic stirring was carried out for reaction at room temperature for 5-10 hours; then, the reaction solution was subjected to rotary evaporation and repeatedly washing with isopropanol, and a final product was dried in a vacuum oven until the mass was constant (25° C.), and finally the hyperbranched polylysine anti-scar compound was obtained.

3 parts of the aloe extract, 7 parts of the cactus extract, 15 parts of the thickener, 0.1 part of the hyperbranched polylysine anti-scar compound prepared above, 10 parts of glycerol and 80 parts of deionized water were weighed respectively and were fully stirred and dissolved uniformly for later use; 2.0 parts of carbomer 940 were added into 80 parts of water, the mixture was heated to 60° C. to fully dissolve carbomer 940 in water, and then cooled to room temperature, poured into the prepared thickener solution, and the mixture was fully stirred and dissolved; finally a proper amount of a pH regulator was added to adjust the pH to 6.8-7.2, so as to obtain an antibacterial hydrogel dressing for promoting scarless wound healing.

The hydrogel dressing has good air permeability and moisture retention, and has the characteristics of antibiosis, no sensitization, safety, comfort, nature and environmental protection. The dressing also has antibacterial, anti-inflammatory, wound healing promoting, hemostasis, moisture absorption and moisture retention, scar hyperplasia inhibiting and good biocompatibility. The hyperbranched polylysine anti-scar compound used in this dressing contains a large number of structures with amino terminal groups, which has efficient and safe bactericidal effect and scar inhibition effect.

Under laboratory conditions, the sterilization effects of hydrogel dressings prepared in all examples on Escherichia coli, Staphylococcus aureus, Candida albicans and Pseudomonas aeruginosa were tested. For the specific detection method, refer to Appendix C4 of Hygienic Standard for Disposable Sanitary Articles (15979-2002), and the stock solution of each sample was used to co-culture with bacteria for 16 hours, and the test temperature was 37° C. The generations of Escherichia coli, Staphylococcus aureus, Candida albicans and Pseudomonas aeruginosa were four generations, and a bacterial suspension was prepared with a phosphate buffer solution (PBS) of 0.03 mol/L. At the same time, by targeting downstream proteins, the pathway of fibroblasts was down-regulated, and the fibrosis of wounds was inhibited, so as to achieve the effect of inhibiting scar hyperplasia. According to the present application, a drug with composite functions was prepared by chemically bonding HBPL with an anti-scar drug. On the one hand, HBPL was released from the hydrogel to the wound surface, which could kill the bacteria on the surface, reduce the infection level and promote wound healing; on the other hand, the anti-scar drug can target proteins associated with initiating cellular fibrotic pathways, and salvianolic acid B can target downstream effector protein CD36^{[1]}. the chemically bonded HBPL is delivered to the deep part of the wound, that is, it played an enhanced antibacterial effect on the surface and inside of the wound through diffusion and targeting^{[2]}. In addition, the over-expression of inflammatory factors is another key factor of scar formation, a large number of terminal amino groups contained in HBPL can adsorb inflammatory factors containing acidic groups, which can directly adsorb inflammatory factors at targeted sites, thus reducing the level of inflammation or even eliminating inflammation, thus further improving the scar inhibition function of the dressing. If HBPL and anti-scar drugs are simply physically blended, HBPL cannot achieve the above effects only by acting on the wound surface. In addition, by forming the hyperbranched polylysine anti-scar compound, and through the coordination and regulation of the components, the present application makes full use of the advantages of respective components, and obtains an antibacterial hydrogel dressing with excellent effect of promoting scarless wound healing, which can be directly used before the wound is closed, and can keep the wound moist, promote wound healing and generate no scar.

Antibacterial experiment data are shown in the following table.

| | Antibacteria rates (%) | | | |
|---|---|---|---|---|
| | Escherichia | Staphylococcus | Candida | Pseudomonas |
| Sample | coli | aureus | albicans | aeruginosa |
| Example 1 | ≥99.99 | ≥99.99 | ≥99.99 | ≥99.99 |
| Example 2 | ≥99.99 | ≥99.99 | ≥99.99 | ≥99.99 |
| Example 3 | ≥99.99 | ≥99.99 | ≥99.99 | ≥99.99 |
| Example 4 | ≥99.99 | ≥99.99 | ≥99.99 | ≥99.99 |
| Example 5 | ≥99.99 | ≥99.99 | ≥99.99 | ≥99.99 |
| Example 6 | ≥99.99 | ≥99.99 | ≥99.99 | ≥99.99 |

It can be seen from the data in the above table that the selection of anti-scar drugs and the synthesis method of the compound do not affect the antibacterial performance of the hydrogel dressing prepared by the present application.

Although the embodiments of the present application have been shown and described, it will be understood by those skilled in the art that many changes, modifications, substitutions and variations can be made to these embodiments without departing from the principle and spirit of the present application, and the scope of the present application is defined by the appended claims and their equivalents.

### REFERENCES

[1] M. F. Griffin, M. R. Borrelli, J. T. Garcia, M. Januszyk, M. King, T. Lerbs, L. Cui, A. L. Moore, A. H. Shen, S. Mascharak, N. M. Diaz Deleon, S. Adem, W. L. Taylor, H. E. desJardins-Park, M. Gastou, R. A. Patel, B. A. Duoto, J. Sokol, Y. Wei, D. Foster, K. Chen, D. C. Wan, G. C. Gurtner, H. P. Lorenz, H. Y. Chang, G. Wernig, M. T. Longaker, JUN promotes hypertrophic skin scarring via CD36 in preclinical in vitro and in vivo models, Science Translational Medicine 13 (609) (2021) eabb3312.
[2] X. Liu, Y. Sun, J. Wang, Y. Kang, Z. Wang, W. Cao, J. Ye, C. Gao, A tough, antibacterial and antioxidant hydrogel dressing accelerates wound healing and suppresses hypertrophic scar formation in infected wounds, Bioactive Materials 34 (2024) 269-281.

What is claimed is:

1. An antibacterial dressing for promoting scarless wound healing, wherein the dressing consists of 0.05-0.1 part by weight of a hyperbranched polylysine anti-scar compound, 0.1-2.0 parts by weight of carbomer, 1-10 parts by weight of a thickener, 2-12 parts by weight of a humectant, 150-200 parts by weight of water, and 1-15 parts by weight of a plant extract; a pH is adjusted to 6.8-7.2 by alkali;
a method for preparing the hyperbranched polylysine anti-scar compound is that:
synthesizing a compound of thiolated hyperbranched polylysine and an anti-scar drug through Michael addition, which specifically comprising: taking hyperbranched polylysine to prepare an aqueous solution of 10-30 mg/mL, mixing a fresh stock solution of 10-40 mg/ml of 2-iminothiolane hydrochloride stock solution with the hyperbranched polylysine solution in a volume ratio of 3:1, reacting at room temperature, performing rotary evaporation on the reaction solution, repeating washing with isopropanol and rotary evaporation, and drying in a vacuum oven until a mass is constant; dissolving the obtained thiol-modified hyperbranched polylysine and an anti-scar drug in water in a mass ratio of 1:1 to prepare a solution with a total concentration of 10-20 mg/mL, reacting at 50° C. for 5 hours, performing rotary evaporation on the reaction solution, repeating washing with isopropanol and rotary evaporation, and drying in a vacuum oven until mass is constant, to obtain the hyperbranched polylysine anti-scar compound;
or:
synthesizing a compound of hyperbranched polylysine and an anti-scar drug by catalyzing by 1-ethyl-(3-dimethylaminopropyl) carbodiimide, which specifically comprising: weighing by mass 1-30 parts of hyperbranched polylysine and 1-30 parts of the anti-scar drug, adding into 100 parts of deionized water, and stirring until completely dissolved to obtain a mixed solution; weighing 0.6 part of 1-ethyl-(3-dimethylaminopropyl) carbodiimide and 0.3 part of N-hydroxysuccinimide, adding into the mixed solution, adjusting pH to 5.5 with 2-(N-morpholine) ethanesulfonic acid, and magnetically stirring at room temperature for reaction for 5-10 hours; subjecting the reaction solution to rotary evaporation, and repeating washing with isopropanol and rotary evaporation, and drying obtained product in a vacuum oven until mass is constant, to obtain the hyperbranched polylysine anti-scar compound;
wherein the anti-scar drug is at least one of asiaticoside, salvianolic acid B and papain.

2. The antibacterial dressing for promoting scarless wound healing of claim 1, wherein the thickener is at least one of polyvinyl alcohol, hydroxypropyl methylcellulose, hydroxyethyl cellulose and polyvinylpyrrolidone.

3. The antibacterial dressing for promoting scarless wound healing of claim 1, wherein the humectant is glycerol.

4. The antibacterial dressing for promoting scarless wound healing of claim 1, wherein the plant extract is at least one of aloe extract and cactus extract.

5. The antibacterial dressing for promoting scarless wound healing of claim 1, preparing by the following method:
adding 1-10 parts of the thickener and 2-12 parts of the humectant into 60-90 parts of deionized water, magnetically stirring and dissolving uniformly, adding 1-10 parts of the plant extract, and fully stirring and dissolving in vacuum for later use;
adding 0.1-2.0 parts of carbomer into 90-100 parts of water for vacuum magnetic stirring and dissolution;
uniformly mixing the prepared two solutions, and adding an alkali solution to adjust pH to 6.8-7.2; adding the hyperbranched polylysine anti-scar compound, magnetically stirring and mixing uniformly, and centrifuging to defoam to obtain the antibacterial dressing for promoting scarless wound healing.

6. The antibacterial dressing for promoting scarless wound healing of claim 5, wherein the alkali is at least one of triethanolamine, sodium hydroxide and phosphate buffer.
